(19) United States
(12) Patent Application Publication
(10) Pub. No.: US 2010/0267968 A1
(43) Pub. Date: Oct. 21, 2010

(54) METHOD FOR THE PREPARATION OF DULOXETINE HYDROCHLORIDE

(75) Inventors: Mahender Rao Siripragada, Mohali (IN); Arulmoli Thangavel, Bangalore (IN); Muthulingham Arunagiri, Bangalore (IN); Prasadachari Yarroju, Hyderabad (IN); Kiranmye Tayyala, Chennai (IN)

Correspondence Address: OLIFF & BERRIDGE, PLC, P.O. BOX 320850, ALEXANDRIA, VA, 22320-4850, US

(73) Assignee: ORCHID CHEMICALS & PHARMACEUTICALS LIMITED, CHENNAI, TAMILNADU (IN)

(21) Appl. No.: 12/734,580

(22) PCT Filed: Dec. 26, 2008
(86) PCT No.: PCT/IB2008/003636

§ 371 Date: Jun. 28, 2010

(30) Foreign Application Priority Data
Dec. 26, 2007 (IN) 3097/CHE/2007

(51) Int. Cl. C07D 333/20

(52) U.S. Cl. 549/75

(57) ABSTRACT

The present invention relates to an improved process for the preparation of Duloxetine and its intermediates (S)-(+)-N,N-dimethyl-3-(1-naphthalenyloxy)-3-(2-thienyl)propanamine by reacting (S)-(+)-N,N-dimethyl-3-(2-thienyl)-3-hydroxypropanamine with 1-fluoronaphthalene in the presence of a base; wherein the improvement lies in conducting the reaction in the absence of solvent.

The following specification describes the nature of the invention; and the manner in which it has to be performed:

### FIELD OF THE INVENTION

The present invention relates to an improved process for the preparation of intermediate used in the preparation of Duloxetine or its pharmaceutically acceptable salts. More particularly the present invention relates to an improved process for the preparation of (S)-(+)-N,N-dimethyl-3-(1-naphthalenyloxy)-3-(2-thienyl)propanamine, represented by the formula (II).

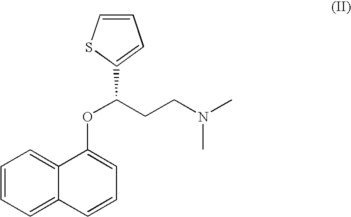

### BACKGROUND OF THE INVENTION

Duloxetine hydrochloride marketed as CYMBALTA® in USA is a serotonin-norepinephrine reuptake inhibitor (SNRI) used for major depressive disorder (MDD), generalized anxiety disorder (GAD), pain related to diabetic neuropathy and fibromyalgia and in some countries for stress urinary incontinence (SUI). Duloxetine hydrochloride is chemically known as (+)-(S)—N-methyl-γ-(1-naphthyloxy)-2-thiophenepropylamine hydrochloride represented by the formula (I).

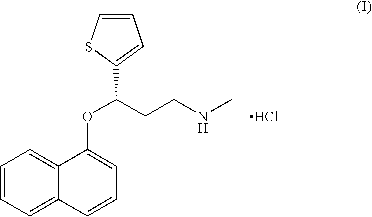

Duloxetine was first disclosed in U.S. Pat. No. 4,956,388 by Robertson, et al., and its synthesis was discussed in more detail by Deeter, et al., in Tetrahedron Letters, 31 (49), 7101-04 (1990).

U.S. Pat. No. 5,023,269 patent, a divisional patent of U.S. Pat. No. 4,956,388 claims Duloxetine and its pharmaceutically acceptable salts. The patent also discloses a process for the preparation of salts of duloxetine via the formation of (S)-(+)-N,N-dimethyl-3-(1-naphthalenyloxy)-3-(2-thienyl)propanamine oxalate. The (S)-(+)-N,N-dimethyl-3-(1-naphthalenyloxy)-3-(2-thienyl)propanamine is prepared by reacting α-2-(dimethylamino)ethyl]-2-thiophene methanol with 1-Fluoronaphthalene in presence of sodium hydride in dimethylacetamide as a solvent.

U.S. Pat. No. 5,362,886 patent provides a process where the (S)-(+)-N,N-dimethyl-3-(1-naphthalenyloxy)-3-(2-thienyl)propanamine is prepared by the dissolving (S)-(−)-N,N-dimethyl-3-(2-thienyl)-3-hydroxypropanamine with sodium hydride in dimethylsulfoxide (DMSO) and reacting with 1-fluoronaphthalene in the presence of potassium salts, such as potassium benzoate or potassium acetate.

U.S. Pat. No. 6,541,668 claims a process of reacting (S)-(−)-N,N-dimethyl-3-(2-thienyl)-3-hydroxypropanamine with 1-fluoronaphthalene in the presence of 1,3 dimethyl-2-imidazolidinone or N-methylpyrrolidine as a solvent at a temperature of about 140° C. The publication also discloses the use of potassium t-butoxide during the condensation reaction.

PCT publication WO 2006/126213 (623/CHE/2005) discloses a method of preparation of compound of formula II by condensing (S)-(−)-N,N-dimethyl-3-(2-thienyl)-3-hydroxypropanamine with 1-fluoronaphthalene in the presence of an alkoxide in an organic polar solvent selected from Dimethyl sulphoxide, N,N-dimethyl formamide and N,N-dimethyl acetamide.

US 20070238883 (IN 7033/DELNP/2008) claims a process for the preparation of (S)-(+)-N,N-dimethyl-3-(1-naphthalenyloxy)-3-(2-thienyl)propanamine by reacting S-(−)—N,N-dimethyl-3-Hydroxy-3-(2-thienyl)propanamine with a base selected from the group consisting of: alkali metal hydroxide, sodium metal alkoxides, lithium metal alkoxides, and 1-fluoronaphthalene, in a polar aprotic solvent selected from the group consisting of: C₅-C₈ aromatic hydrocarbons, ionic liquid, dimethyl Sulfoxide (DMSO), dimethylformamide (DMF), dimethylacetamide (DMA), acetonitrile, sulfolane, nitromethane, propylene carbonate and mixtures, wherein the reaction is conducted in the absence of a phase transfer catalyst.

WO 2007/096707 claims a process for preparation of Duloxetine or its intermediate by reacting (S)-3-(dimethylamino)-1-(2-thienyl)-1-propanol and 1-fluoronaphthalene and at least one alkaline metal hydroxide or alkoxide in DMSO or DMSO-cosolvent mixtures, where the process does not require the use of a phase transfer catalyst.

WO 2007/045405 publication claims a reaction between 1-fluoronaphthalene and (S)-(−)-3-N,N-dimethylamino-1-(2-thienyl)-propan-1-ol in presence of 1,3-dimethyl-2-oxo-hexahydropyrimidine as the solvent to give ((+)-(S)—N,N-dimethyl-γ-(1-naphthalenyloxy)-2-thiophenepropanamine) and then converting to duloxetine.

In the prior art processes, coupling of (S)-(−)-N,N-dimethyl-3-(2-thienyl)-3-hydroxypropanamine with 1-fluoronaphthalene is conducted in presence of a strong base, such as sodium hydride, in a protic polar solvent, such as DMSO, DMAC etc. The dimesyl anion generated on the use of DMSO in the prior art reaction conditions makes the reaction hazardous due to non-compatibility of DMSO with strong bases (NaH, NaOH etc) and also results in the formation of impurity or by-products in the form of R-isomer; whereby partial or complete racemisation of the condensed product take place.

The solvents may also be carried till the end of the reaction and remain as residual solvents in the final API. The removal of these solvents is difficult and more workup has to be done in order to obtain a product of high purity. Thus if the condensation reaction is performed in such a solvent system, the final API obtained would be of low quality and in lower yield. Similar is the case with DMF and DMAc.

The use of the high boiling solvents like DMSO, DMF, DMAc as mentioned in the prior art for the condensation of (S)-(−)-N,N-dimethyl-3-(2-thienyl)-3-hydroxypropanamine and 1-fluoronaphthalene is difficult to recover and hence industrially not viable. Further use of NaH along with DMSO found to be hazardous and hence industrially not feasible.

In our continued research for developing a process for the preparation of Duloxetine or its pharmaceutically acceptable salts; we have identified a convenient process which avoids the use of the hazardous and expensive solvents for the nucleophilic aromatic displacement reaction between 1-fluoronaphthelene and (S)-3-N,N-dimethylamino-1-(2-thienyl)-propan-1-ol. The process of the present invention reduces the formation of impurity or by-product and improves the quality and yield of the product and eliminates the foregoing problems associated with prior art processes. The process of the present invention is also an industrially scalable and economically viable process.

### OBJECTIVE OF THE INVENTION

The main objective of the present invention is to provide a simple and a safe process for the preparation of thiophene derivative of formula II, which is a commercially viable and an industrially scalable process

Another objective of the present invention is to provide an improved simple process for the preparation of Duloxetine hydrochloride of high purity and quantity.

### SUMMARY OF THE INVENTION

Accordingly the present invention provides a process for the preparation of Duloxetine hydrochloride of formula (I) as provided in the scheme (I)

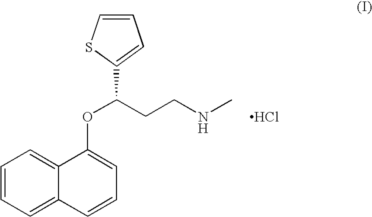

comprising the steps of:
(i) reacting (S)-(−)-N,N-dimethyl-3-(2-thienyl)-3-hydroxypropanamine of formula (III) with 1-fluoronaphthalene of formula (IV) in the presence of a base; wherein the improvement lies in conducting the reaction in the absence of solvent;
(ii) optionally converting (S)-(+)-N,N-dimethyl-3-(1-naphthalenyloxy)-3-(2-thienyl)propanamine (in situ) to (S)-(+)-N,N-dimethyl-3-(1-naphthalenyloxy)-3-(2-thienyl) propanamine acid addition salt of formula (V) and hydrolyzing the compound of formula (V) using alkali metal hydroxide and in presence of a solvent;
(iii) demethylation of the resulting compound to Duloxetine base using phenylchloroformate and a base in presence of a solvent; and
(iv) conversion of Duloxetine base of formula (VII) to its pharmaceutically acceptable salt of formula (I).

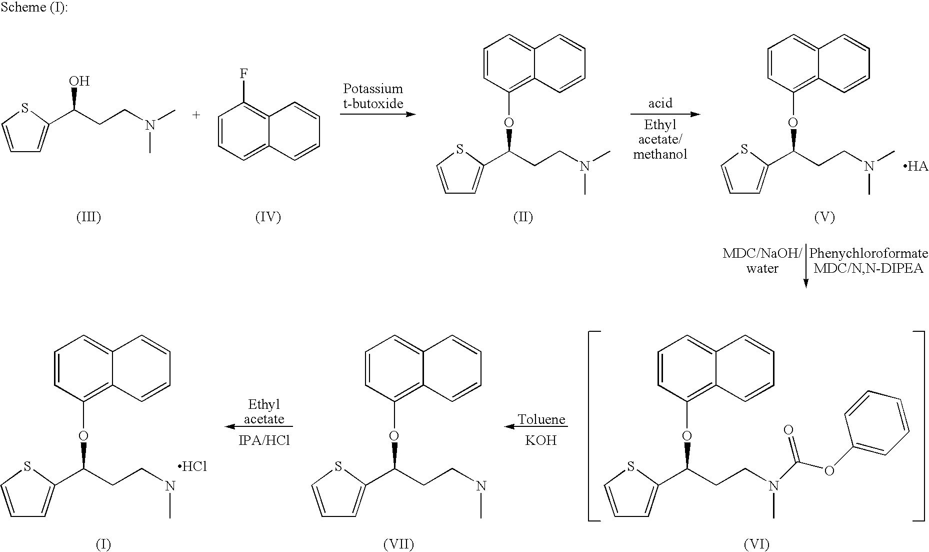

### DETAILED DESCRIPTION OF THE INVENTION

In one embodiment of the present invention the reaction between (S)-(−)-N,N-dimethyl-3-(2-thienyl)-3-hydroxypropanamine and 1-fluoronaphthalene is conducted in the absence of a solvent to overcome the problems associated with the prior art process as discussed above.

In an embodiment of the present invention the step (i) reaction i.e. the reaction between (S)-(−)-N,N-dimethyl-3-(2-thienyl)-3-hydroxypropanamine and 1-fluoronaphthalene is conducted in the presence of a base selected from alkali metal hydroxides or alkaline earth metal hydroxide comprising potassium hydroxide, sodium hydroxide, calcium hydroxide, magnesium hydroxide and the like or an alkoxide selected from alkali metal alkoxide comprising lithium, sodium, and potassium alkoxide and the like, more preferably potassium t-butoxide.

In another embodiment of the present invention the (S)-(+)-N,N-dimethyl-3-(1-naphthalenyloxy)-3-(2-thienyl)propanamine obtained in step (i) is optionally converted into its acid addition salts [.HA] selected from oxalate, phosphate, or hydrochloride salt; preferably oxalate salt of (S)-(+)-N,N-dimethyl-3-(1-naphthalenyloxy)-3-(2-thienyl)propanamine is employed.

In yet another embodiment of the present invention, the acid addition salts of (S)-(+)-N,N-dimethyl-3-(1-naphthalenyloxy)-3-(2-thienyl)propanamine is prepared by dissolving the base of formula (II) and reacting with the corresponding acid, preferably oxalic acid isolating the acid addition salt of formula (V) using an alcohol selected from C₁₋₅ alkanol, preferably methanol.

In another embodiment of the present invention the hydrolysis step (iii) is conducted in presence of alkali metal hydroxide or alkaline earth metal hydroxide selected from potassium hydroxide, sodium hydroxide, calcium hydroxide, magnesium hydroxide and a solvent selected from water, chlorinated solvents comprising dichloromethane, chloroform or dichloroethane and the like; aromatic hydrocarbons comprising toluene, xylene, benzene and the like or mixtures there of.

In another embodiment of the present invention the demethylation step (iv) is carried out in presence of N,N diisopropylethylamine and a solvent selected from water, chlorinated solvent comprising of dichloromethane, chloroform, or dichloroethane and the like or mixtures there of to obtain the carbamate, which is then hydrolyzed to duloxetine free base in presence of an alkali metal hydroxide and a solvent selected from toluene, benzene, xylene, dichloromethane chloroform and the like or mixtures there of.

In another embodiment of the present invention the duloxetine free base of formula (VII) is converted to duloxetine hydrochloride by adjusting the pH of the solution to 5.3-5.8 with concentrated HCl in isopropyl alcohol in a solvent selected from ethyl acetate.

The starting material of the reaction was prepared as per the process known in the prior art. The (S)-(−)-N,N-dimethyl-3-(2-thienyl)-3-hydroxypropanamine one of the key intermediate/starting material in the preparation of duloxetine was prepared by reacting 2-acetylthiophene with formaldehyde and N,N-dimethylamine hydrochloride in an alcoholic solvent like ethanol, isopropanol, isobutyl alcohol to obtain 3-(dimethylamino)-1-(2-thienyl)-1-propanone hydrochloride which is then reduced to its alkanol followed by resolution with mandelic acid.

The other key starting material 1-fluoronaphthalene was prepared from 1-aminonaphthalene by diazotization with NaNO₂ in HCl and treating with HBF₄. The diazonium fluoroborate was collected and washed with ethanol and decomposed to crude fluoronaphthalene by heating, which was then extracted with sodium hydroxide and distilled to obtain pure 1-fluoronaphthalene.

The present invention is exemplified by the following examples, which are provided for illustration only and should not be construed to limit the scope of the invention.

### Example (1)

### Preparation of (S)-(+)-N,N-dimethyl-3-(1-naphthalenyloxy)-3-(2-thienyl) propanamine oxalate

1-Fluoronaphthalene (118.3 g) and (S)-(−)-N,N-dimethyl-3-(2-thienyl)-3-hydroxypropanamine (100 g) was taken in a 500 mL round bottom flask and stirred for 10 minutes. Powdered potassium tertiary butoxide was added to the reaction mass, heated to 90-100° C. and maintained for 20-22 hrs for the completion of the reaction. After completion of the reaction; the reaction mass was cooled and to it was added toluene and stirred. The layers were separated and the aqueous layer was extracted with toluene. The combined toluene layer was washed with water followed by 5% HCl solution. The acidic aqueous layer was extracted with dichloromethane and combined organic layer was washed with 5% sodium hydroxide then with water. The organic layer was distilled atmospherically and finally under vacuum to get the thick mass. Ethyl acetate was added to the thick mass and distilled out dichloromethane completely under vacuum and stirred the reaction mass followed by the addition of ethyl acetate under stirring at 25-30° C. A solution of methanol and oxalic acid were added to the reaction mass at 25-30° C. and stirred for 60-90 minutes at 0-5° C. The solid was filtered washed with cold ethyl acetate twice and dried to yield the titled compound. Yield 154.g

### Example (2)

### Preparation of Duloxetine

To (S)-(+)-N,N-dimethyl-3-(1-naphthalenyloxy)-3-(2-thienyl)propanamine oxalate was added aqueous sodium hydroxide and stirred. To the reaction mass was added dichloromethane under stirring and the layers were separated. The aqueous layer was extracted with dichloromethane and washed with water. The organic layer was distilled to get thick mass.

To the thick mass was added dichloromethane and the contents was heated to 40° C. under azeotropic condition. The dichloromethane was distilled off and the reaction mass was cooled to 25-30° C. To the above mass was added N,N-diisopropylethylamine and Phenyl chloroformate, stirred and heated for the completion of the reaction. After completion of reaction, water was added to the reaction mass and the layers were separated. The aqueous layer was extracted with dichloromethane. The collective organic layer was washed with 5% sodium bicarbonate solution and then water. The organic layer was distilled at 40° C. and finally under vacuum to get the thick mass and it was cooled to 25-30° C.

To the thick mass obtained above was added toluene and Potassium hydroxide and heated to 110° C. and stirred for few hours at the same temperature. After completion of the reaction; the reaction mass was cooled and filtered through hyflow bed. The hyflow bed was washed with toluene and collective toluene layer was washed with water. The combined toluene layer was washed with 5% HC1 solution and the acidic aqueous layer was extracted with dichloromethane. The organic layer was washed with 5% sodium hydroxide solution then with water. The organic layer was distilled to get the thick mass. To the thick mass was added ethyl acetate solution and dichloromethane was distilled off completely under vacuum to obtain a thick mass of duloxetine base.

### Example 3

### Preparation of Duloxetine Hydrochloride

A solution of duloxetine base in ethyl acetate was subjected to carbon treatment, followed by the adjusting the pH of the solution to 5.3-5.8 with the solution of hydrochloric acid in isopropyl alcohol. The crude solid was purified by refluxing in ethyl acetate and methanol solution and cooling to crystallize the product washed with ethyl acetate to obtain pure duloxetine hydrochloride.

1. A process for preparation of (S)-(+)-N,N-dimethyl-3-(1-naphthalenyloxy)-3-(2-thienyl) propanamine of formula (II) or its acid addition salt 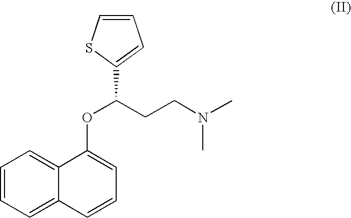
comprising reacting (S)-(−)-N,N-dimethyl-3-(2-thienyl)-3-hydroxypropanamine of formula (III) 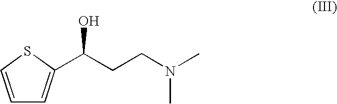
with 1-fluoronaphthalene of formula (IV) 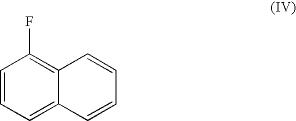
in the presence of a base; wherein the improvement lies in conducting the reaction in the absence of solvent.

2. A process according to claim 1, wherein the base is selected from alkali metal hydroxide or alkaline earth metal hydroxide comprising potassium hydroxide, sodium hydroxide, calcium hydroxide, magnesium hydroxide or from alkali metal alkoxide comprising lithium, sodium, and potassium alkoxide.

3. A process according to claim 1, wherein the acid addition salts of (S)-(+)-N,N-dimethyl-3-(1-naphthalenyloxy)-3-(2-thienyl)propanamine is selected from oxalate, phosphate or hydrochloride salt.

4. A process for preparation of duloxetine or its acid addition salt, the process comprising:
reacting (S)-(±)-N,N-dimethyl-3-(1-naphthalenyloxy)-3-(2-thienyl) propanamine of formula (II) or its acid addition salt 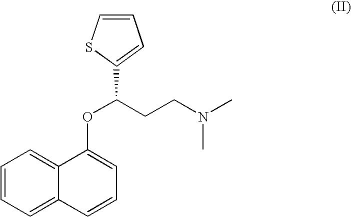
in the presence of a base

5. A process according to claim 1, further comprising the steps of:
(i) hydrolysis of formula (V) in presence of a base and a solvent; 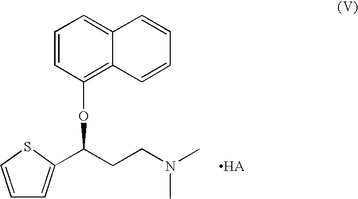
(ii) demethylating the resulting compound; and
(iii) optionally converting the demethylated compound to its acid addition salt.

6. A process according to claim 5, wherein the hydrolysis step (i) is performed in presence of an alkali metal hydroxide or alkaline earth metal hydroxide selected from potassium hydroxide, sodium hydroxide, calcium hydroxide, magnesium hydroxide and a solvent selected from water, dichloromethane, dichloroethane, chloroform, ethyl acetate, toluene, xylene, benzene and mixtures thereof.
